(19) United States
(12) Patent Application Publication
(10) Pub. No.: US 2015/0367137 A1
(43) Pub. Date: Dec. 24, 2015

(54) SYSTEM AND METHOD FOR REMOTELY CONTROLLING AN IMPLANTABLE NEUROSTIMULATOR

(71) Applicant: Will Rosellini, Dallas, TX (US)

(72) Inventor: Will Rosellini, Dallas, TX (US)

(73) Assignee: REMOTE BIOMEDICAL LLC, Dallas, TX (US)

(21) Appl. No.: 14/309,926

(22) Filed: Jun. 20, 2014

(51) Int. Cl. A61N 1/372; A61N 1/36

(52) U.S. Cl. CPC A61N 1/37252; A61N 1/36067; A61N 1/36128; A61N 1/37235; A61N 1/37282; A61N 1/37217

(57) ABSTRACT

The present invention relates a system for remote monitoring and programming of an implantable neurostimulator for deep brain stimulation, comprising: an implantable neurostimulator configured to stimulate at least one target site of brain; an external wireless device in communication with the implantable neurostimulator; and a remote mobile device configured to communicate and exchange data with the implantable neurostimulator through the external wireless device over a communication network.

### FIELD OF THE INVENTION

The present invention relates to remotely controlling an implantable medical device, in particular, to system and method for remote programming and monitoring of an implantable neurostimulator device for deep brain stimulation over a communication network.

### BACKGROUND OF THE INVENTION

Parkinson's disease (PD) is characterized by unnatural motor movements. Most frequently these symptoms are manifested in the form of tremor, bradykinesia and/or rigidity of a subject's upper extremities. However, other symptoms associated with PD include negative effects on gait, balance, speech, olfaction, sleep and cognition. These symptoms are partly responsible for the subject's functional disability and social embarrassment. Various treatments have been developed to alleviate many of the symptoms of PD. The treatments can involve pharmaceutical interventions, fetal cell transplants, surgery, or electrical stimulation, such as deep brain stimulation (DBS) or functional electrical stimulation (FES), in some of these disorders.

The most commonly used treatment option for PD is deep brain stimulation (DBS). During the last two decades, more than 100,000 patients worldwide have been treated with DBS for movement disorders. Deep brain stimulation (DBS), such as of the thalamus or basal ganglia, is also a clinical technique for the treatment of movement disorders such as essential tremor, myoclonus dystonia syndrome (MDS) and other physiological disorders. DBS may also be useful for traumatic brain injury and stroke.

The electric parameters of implantable pulse generator (IPG) are set and monitored by the clinicians using the clinical programmer, allowing for different stimulation parameters. The parameter settings are patient specific and may be changed at any programming session to optimize the patient's symptom relief. A handheld therapy controller provided along to the patient allows to switch the device on and off, as well as to change stimulation intensity within a window of parameters decided by the clinician during the programming session. It also shows charging status and battery level of IPG.

DBS devices typically comprise a very thin insulated wire lead terminated with four electrode contacts. The lead is routed out of the skull through a small opening and connected to an extension wire subcutaneously routed along the head, neck, and shoulder to an impulse generator or other suitable neurostimulator device implanted under the skin, for example, in the chest area. As such, conventional DBS procedures and devices require two surgical procedures: a surgical procedure to implant the electrodes within the brain, and a second surgical procedure to implant the neurostimulator device in the chest.

Recent studies show that the patients relied on medical staff for every aspect of operation and handling of the implanted device using therapy controller. In addition to the initial programming, a majority of patients left all subsequent adjustments or handling of the device in the care of their nurse, neurologist, or neurosurgeon. Besides initial programming, a majority of patients had to visit the hospital for subsequent tuning of the stimulation parameters on several occasions which proved cumbersome and expensive.

In addition, most of the DBS treated patients did not want to manage the device themselves in terms of changing electric settings within their preset window of parameters.

On the other hand, the patients felt secure and in control in being able to check the battery level using the handheld controller. Some patients who had recognized a depleted IPG did so only by the return of symptoms of the disease or due to vanishing side-effects of DBS.

US patent publication US20090287273 A1 describes a clinical programmer system interface for monitoring patient progress. PCT publication WO2013012625 A1 discloses movement disorder monitoring system and method for continuous monitoring. U.S. Pat. No. 8,412,332 B2 discloses a miniature wireless system for deep brain stimulation. U.S. Pat. No. 8,485,979 B2 shows a system and method for monitoring or treating nervous system disorders.

A medical device system that provides therapy treatment for a nervous system disorder may support a plurality of features that are associated with the therapy treatment. However, additional features may be added to the medical device system in order to enhance an existing functionality or to provide an additional functionality. Consequently, there is a need for remote programming and monitoring of the implanted neurostimulator for deep brain stimulation.

### SUMMARY OF THE INVENTION

The present invention discloses systems and methods for remote monitoring and programming of an implantable neurostimulator device for deep brain stimulation over a communication network in patients with neurological disorders.

In one embodiment, the present invention discloses a system for remote controlling of an implantable neurostimulator for deep brain stimulation, comprising: an implantable neurostimulator configured to stimulate at least one target site of brain; an external wireless device for networking over a network and in communication with the implantable neurostimulator; and a remote mobile device configured to communicate and exchange data with the implantable neurostimulator over the network.

In another embodiment, the present invention discloses a method of remote monitoring and programming of an implantable neurostimulator, the method comprising following steps : i) providing an implantable neurostimulator for stimulating a nerve site; ii) providing an external wireless device in communication with the implantable neurostimulator; iii) providing a remote mobile device for networking over a communication network; and iv) establishing a communication and exchanging data between the remote mobile device and implantable neurostimulator through external wireless device over the communication network.

Still other embodiments of the present invention will become readily apparent to those skilled in the art from the following detailed description, wherein are described embodiments by way of illustrating the best mode contemplated for carrying out the invention. As will be realized, the invention is capable of other and different embodiments and its several details are capable of modifications in various obvious respects, all without departing from the spirit and the scope of the present invention. Accordingly, the drawings and detailed description are to be regarded as illustrative in nature and not as restrictive.

### BRIEF DESCRIPTION OF THE DRAWINGS

FIG. 1 illustrates a block diagram of a system for remote controlling of an implantable neurostimulator according to an embodiment of the present invention.

FIG. 2 illustrates a flow diagram of a method of remote monitoring and programming of an implantable neurostimulator according to an embodiment of the present invention.

FIG. 3 illustrates a block diagram of a system for remote monitoring and programming of an implantable neurostimulator according to an embodiment of the present invention.

### DETAILED DESCRIPTION OF THE INVENTION

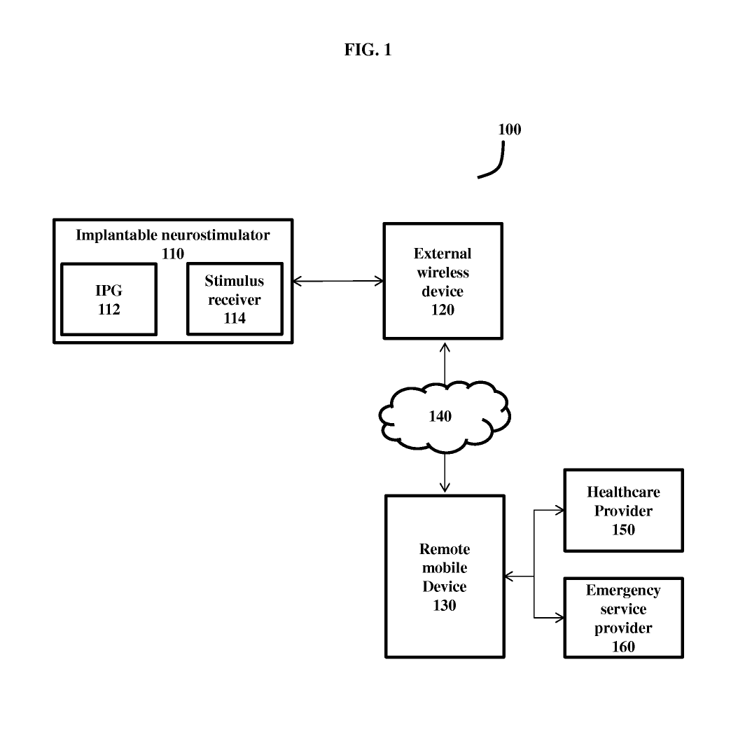

FIG. 1 is a block diagram that schematically illustrates a system 100 for for remote controlling of an implantable neurostimulator in accordance with an embodiment of the present invention. The system 100 comprises an implantable neurostimulator 110 configured to stimulate one or more regions of brain during deep brain stimulation. The implantable neurostimulator 110 is inductively coupled with an external wireless device 120, which is capable of bidirectional communication with the implantable neurostimulator 110 and adapted to networking with a communication network 140. The system further comprises a remote mobile device 130 establishing communication and exchanging data with the implantable neurostimulator 110 through the external wireless device 120 over the communication network 140.

In an embodiment, the implantable neurostimulator 110 comprises an implantable pulse generator 112 for generating electrical impulses at a programmed frequency that is required for neurostimulation. It also comprises an implanted stimulus receiver 114 for receiving external stimulus signals and is capable of applying electrical pulses independently of the pulse generator 112.

The external wireless device 120 located external to the patient' body is inductively coupled and in bidirectional communication with the implantable neurostimulator 110. The external wireless device 120 is adapted to form networking with a communication network 140 and exchanges data related to stimulation parameter, stimulation schedule, patient history, patient health status and treatment details with a remote mobile device 130 upon establishment of connection over the network 140.

The remote mobile device 130 may comprise a desktop computer, a laptop computer, a tablet PC, internet enabled personal digital assistant (PDA), a mobile phone, a pocket PC, and the like. The remote mobile device 130 establishes connection and exchanges data with the implantable neurostimulator 110 through the external wireless device 120 over a communication network 140, which comprises internet, wide area network (WAN), wireless network and virtual private network (VPN). Thus, a physician or a healthcare provider 150 can monitor and program or reprogram the implantable neurostimulator 110 using a system such as a computer from a remote location through the network 140. In an embodiment, the remote mobile device 130 is further connected to an emergency service provider 160, so that during emergency situations such as when the patient experiences a fall or, when there is a marked deviation in stimulation parameters, the emergency service provider 160 is alerted through messages or signals requesting for emergency response.

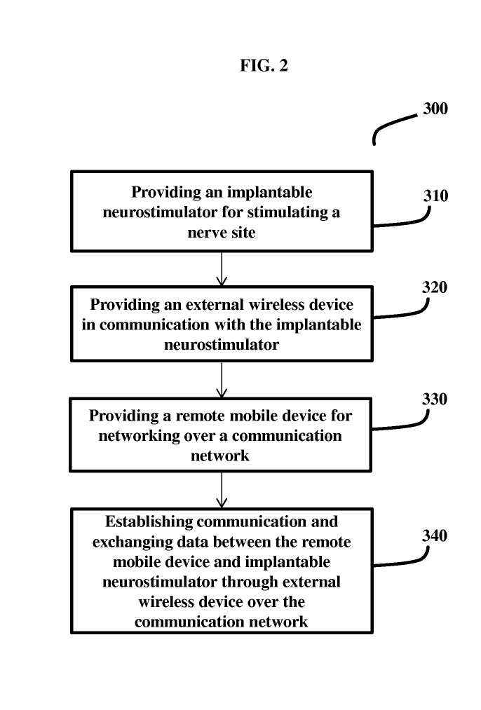

FIG. 2 shows a flow diagram of a method 300 of programming and monitoring an implantable neurostimulator, the method comprising following steps : i) step 310 shows providing an implantable neurostimulator for stimulating a nerve site; ii) step 320 shows providing an external wireless device in communication with the implantable neurostimulator; iii) step 330 shows providing a remote mobile device for networking over a communication network; and iv) step 340 shows establishing communication and exchanging data between the remote mobile device and implantable neurostimulator through external wireless device over the communication network.

The communication connection between the remote mobile device 130 and the implantable neurostimulator 110 via external wireless device may be initiated by a physician or a patient. The physician can interrogate or reprogram the implantable neurostimulator 110 from a remote site utilizing a network 140 including a virtual private network (VPN) thus establishing a secure private connection for exchange of data between physician's computer system or mobile electronic device and the implantable neurostimulator 110.

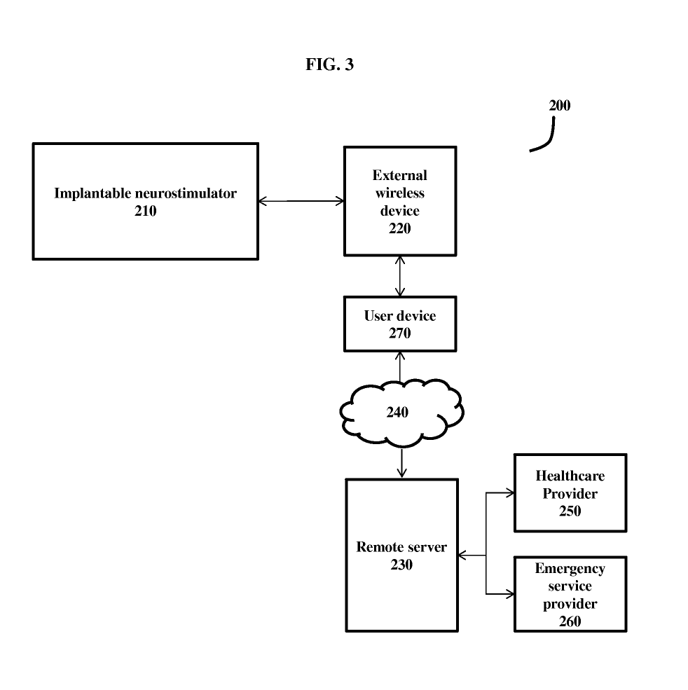

In an embodiment, FIG. 3 shows a system 200 for remote monitoring and programming of an implantable neurostimulator 210 comprising an external wireless device 220 that is connected to a user device 270 such as a computer, a laptop PC, a tablet or a mobile phone which is capable of networking with a communication network 240 and the user device 270 establishes communication, exchanges data with a remote server 230 through a communication network 240. The remote server 230 is further accessed by a healthcare provider 250 and an emergency service provider 260 from a remote location thus offering medical services including programming and monitoring of an implantable neurostimulator 210.

In another embodiment, the remote server 230 provides automatic feedback to the implantable neurostimulator 220 in a closed loop control system based on data received from the external wireless device 220. Those skilled in the art will appreciate that any of a wide variety of stimulation parameters may be monitored and used to implement the closed-loop control system described herein.

What is claimed is:

1. A system for remote controlling of an implantable neurostimulator for deep brain stimulation, comprising:
an implantable neurostimulator configured to stimulate at least one target site of brain;
an external wireless device for networking over a network and in communication with the implantable neurostimulator; and
a remote mobile device configured to communicate and exchange data with the implantable neurostimulator over the network.

2. The system of claim 1, wherein the implantable neurostimulator comprises an implanted pulse generator and an implanted stimulus receiver.

3. The system of claim 1, wherein the remote mobile device comprises a desktop computer, a laptop computer, internet enabled personal digital assistant, a tablet PC and a mobile phone.

4. The system of claim 1, wherein the data exchange comprises exchange of data related to neurostimulation parameters, stimulation schedules, patient information and neurostimulation software.

5. The system of claim 1, wherein external wireless device is in communication with the implantable neurostimulator through a medical implant communication service.

6. The system of claim 1, wherein the network comprises a virtual private network, internet network and wide area network.

7. The system of claim 1, wherein the remote mobile device is further connected to a healthcare provider or emergency medical service provider.

8. A method of remote monitoring and programming of an implantable neurostimulator, the method comprising:
providing an implantable neurostimulator for stimulating a nerve site;
providing an external wireless device in communication with the implantable neurostimulator;
providing a remote mobile device for networking over a communication network; and
establishing a communication and exchanging data between the remote mobile device and the implantable neurostimulator through the external wireless device over the communication network.

9. The method of claim 8, wherein the implantable neurostimulator comprises an implanted pulse generator and an implanted stimulus receiver.

10. The method of claim 8, wherein the remote mobile device comprises a desktop computer, a laptop computer, internet enabled personal digital assistant, a tablet PC and a mobile phone.

11. The method of claim 8, wherein the data exchange comprises exchange of data related to neurostimulation parameters, stimulation schedules, patient information and neurostimulation software.

12. The method of claim 8, wherein external wireless device is in communication with the implantable neurostimulator through a medical implant communication service.

13. The method of claim 8, wherein the network comprises a virtual private network, internet network and wide area network.

14. The method of claim 8, wherein the remote mobile device is further connected to a healthcare provider or emergency medical service provider.
